(19) United States
(12) Patent Application Publication
(10) Pub. No.: US 2018/0071429 A1
(43) Pub. Date: Mar. 15, 2018

(54) SEALANT FORMULATIONS AND USES THEREOF

(71) Applicant: Omrix Biopharmaceuticals Ltd., Rehovot (IL)

(72) Inventors: Ronen EAVRI, Binyamina (IL); Amatzia GANTZ, Givatayim (IL); Roni MINTZ, Moshav Shafir (IL); Israel NUR, Nes Ziona (IL); Damian VISNOVEZKY, Tel Aviv-Yafo (IL)

(21) Appl. No.: 15/700,693

(22) Filed: Sep. 11, 2017

Related U.S. Application Data
(60) Provisional application No. 62/394,366, filed on Sep. 14, 2016.

(30) Foreign Application Priority Data
Sep. 14, 2016 (IL) 247821

(51) Int. Cl. A61L 24/10; A61L 24/04

(52) U.S. Cl. CPC A61L 24/106; A61L 24/043; A61L 2300/254; A61L 2300/418; A61L 2400/04

(57) ABSTRACT

The invention relates to a storage-stable aqueous sealant formulation comprising a blood derived fibrinogen concentrate, a divalent cation and factor XIa.

### TECHNOLOGICAL FIELD

The present disclosure relates to a fibrinogen-based sealant or to a fibrin sealant formulation.

### BACKGROUND ART

References considered to be relevant as background to the presently disclosed subject matter are listed below:

Acknowledgement of the above references herein is not to be inferred as meaning that these are in any way relevant to the patentability of the presently disclosed subject matter.

U.S. Pat. No. 5,985,315

International patent application publication No. WO 93/05822

U.S. Pat. No. 5,792,835

U.S. Pat. No. 6,916,911

U.S. Pat. No. 8,962,033

European Patent No. 1 351 705.

### BACKGROUND

Biological glues/sealants are described in the art. For example, a two component glue is disclosed in U.S. Pat. No. 5,985,315. Specifically, U.S. Pat. No. 5,985,315 describes a method of preparing a blood plasma derived biological glue comprising fibrinogen and at least one coagulation factor, which, in aqueous solution does not coagulate spontaneously, and which is able to coagulate by adding a component containing calcium ions.

In addition, PCT application publication No. WO 93/05822 describes a tissue glue comprising a whole blood cryoprecipitate and a proteolytic enzyme.

U.S. Pat. No. 5,792,835 describes a method of preparing a topical fibrinogen complex from plasma comprising fibrinogen, Factor II, and a low amount of plasminogen.

U.S. Pat. No. 6,916,911 describes a method of preparing fibrin sealant comprising fibrinogen multimers, having at least 6 fibrinogen units.

U.S. Pat. No. 8,962,033 describes the preparation of a fibrin matrix and specifically a method for applying fibrin matrix onto a leaking tissue, the method comprising applying an amount of a solid fibrin sealant blend onto the tissue, the solid blend comprising a proteolytic enzyme capable of forming fibrin when it reacts with fibrinogen, and then applying onto the solid fibrin sealant an amount of a liquid fibrin sealant. The combination of the solid fibrin and the liquid fibrin sealant forms a fibrin matrix on the tissue.

Finally, European Patent No. 1 351 705 describes the combination of recombinant factor VIIa and fibrinogen as effective for treatment of bleeding following intravenous administration of the combination.

### GENERAL DESCRIPTION

The present disclosure is based on the development of a one/single component sealant (glue) formulation comprising fibrinogen, calcium ion and factor XIa (herein also referred to by the abbreviation FXIa). It has been surprisingly found that the combination of these ingredients, as a single component sealant, is storage stable and yet, upon contact with blood, it spontaneously coagulates.

In line with the findings, the present disclosure provides, in accordance with a first of its aspects, a storage-stable aqueous sealant formulation comprising a blood derived fibrinogen concentrate, divalent cation and factor XIa, the sealant formulation is stable at a temperature of between 20° C.-25° C. for at least 5 minutes.

In accordance with a second aspect, there is provided by the present disclosure an applicator comprising (i) a container holding a sealant formulation comprising a storage-stable aqueous sealant formulation comprising a blood derived fibrinogen concentrate, a divalent cation and factor XIa, and the sealant formulation is stable at a temperature of between 20° C.-25° C. for at least 5 minutes; and (ii) a re-sealable opening for delivery therethrough of the sealant formulation.

In accordance with yet a third aspect, there is provided by the present disclosure a wound dressing comprising a support matrix holding a sealant formulation, the sealant formulation comprises storage-stable aqueous sealant formulation comprising a blood derived fibrinogen concentrate, a divalent cation and factor XIa, the sealant formulation is stable at a temperature of between 20° C.-25° C. for at least 5 minutes.

Yet, in accordance with a fourth aspect, there is provided a method for promoting the formation of a fibrin clot, the method comprises contacting blood with a sealant formulation comprising storage-stable aqueous sealant formulation comprising a blood derived fibrinogen concentrate, a divalent cation and factor XIa, and the sealant formulation is stable at a temperature of between 20° C.-25° C. for at least 5 minutes.

Yet further, in accordance with its fifth aspect, the present disclosure provides a method of treating a wound in a subject in need thereof, the method comprises applying onto at least a portion of said wound an amount of a sealant formulation comprising a storage-stable aqueous sealant formulation comprising a blood derived fibrinogen concentrate, a divalent cation and factor XIa, and it is stable at a temperature of between 20° C.-25° C. for at least 5 minutes, the amount of said sealant formulation is effective to promote clotting in said wound when brought into contact with said wound.

The present disclosure provides a kit comprising container including a sealant formulation storage-stable aqueous sealant formulation comprising a blood derived fibrinogen concentrate, a divalent cation and factor XIa, and the sealant formulation is stable at a temperature of between 20° C.-25° C. for at least 5 minutes.

The present disclosure provides a method of manufacturing storage-stable aqueous sealant formulation comprising a blood derived fibrinogen concentrate, a divalent cation and factor XIa, and it is stable at a temperature of between 20° C.-25° C. for at least 5 minutes comprising the steps of:
a) providing a blood derived fibrinogen concentrate ingredient;
b) providing a factor XIa ingredient;
c) providing a divalent cation ingredient; and
d) admixing all the ingredients in order to obtain the formulation.

The term “admixing” means mixing the ingredients in any order, any combination and/or sub-combination.

All embodiments and definitions described herein above and below for the storage-stable aqueous sealant formulation also relate to the applicators, wound dressings, methods and/or kits according to the invention.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is an image showing tested formulations (in vials) and clot formations after addition of Ca²⁺ (vials 1-8, and control vials 9 and 10).

FIG. 2 is a graph showing the effect of CaCl₂ concentrations and different FXIa concentrations on clotting time.

### DETAILED DESCRIPTION OF EMBODIMENTS

The present disclosure provides, in accordance with its first aspect, a storage-stable aqueous sealant formulation comprising a blood derived fibrinogen concentrate, a divalent cation and factor XIa, the sealant formulation is stable at a temperature of between 20° C.-25° C. for at least 5 minutes.

In the context of the present invention, the term “sealant formulation” is to be understood as a single/one component adhesive/glue/hemostat, the formulation having ingredients that upon contact with a tissue and/or blood e.g. in proximity with tissue, react to subsequently form a clot, acting as a tissue adhesive, and thereby stop bleeding, join structures and/or seal physiological leaks, e.g., of cerebrospinal fluids (CSF), lymph, bile, gastrointestinal (GI) content, air leak from lungs etc. In some embodiments, the sealant formulation also comprises therapeutics such that upon natural degradation of the clot formed in the body, the therapeutic is released. The therapeutic can be, without being limited thereto, a drug, such as antibiotics, analgesics, anti-inflammatory drugs, cancer drugs etc, cells including, for example, any type of stem cells e.g. Embryonic Stem (ES) cells, adult stem cells, Pluripotent Stem Cells (iPSCs) etc. from Human or other origin.

At times, the sealant formulation is referred to as a “fibrinogen-based sealant” and in the context disclosed herein it is to be understood as one forming or meaning a fibrin glue, fibrin sealant, fibrin adhesive, fibrin film, fibrin network, fibrin lattice, fibrin mesh, fibrin greed and fibrin gel.

The sealant formulation comprises a blend of at least fibrinogen, divalent cation and Factor XIa. When referring to a “blend” it is to be understood as any form of a mixture, homogenous and non-homogenous mixture of at least the three ingredients. The blend may include other ingredients as further detailed below.

It has been surprisingly found that notwithstanding the presence of a divalent cation in the aqueous formulation, the sealant formulation is inert, i.e. does not spontaneously coagulate for a sufficient time under a selected storage condition. As the sealant formulation is in contact with plasma or blood, Factor XIa activates downstream intrinsic components of the clotting mechanism, which in turn, in the presence of the fibrinogen within the formulation, result in the formation of a glue.

The sealant formulation is an aqueous formulation. When referring to an “aqueous formulation” it is to be understood to encompass a blend of ingredients, in liquid or solid form, that contains water molecule. In some embodiments, the aqueous formulation is in liquid form. When in liquid form, it is in accordance with some embodiments that the liquid carrier is a buffer having an essentially neutral pH, e.g. pH 7.0±0.5.

In some other embodiments, the formulation is in solid form. In some embodiments, the formulation is frozen. Prior to use, the formulation can be thawed.

In yet some other embodiments, the formulation is in a form of a powder e.g. lyophilized and prior to use, or during application onto the target site (e.g. tissue) the powder is moistened with an aqueous, storage suitable, solution or with blood.

The sealant formulation is storage-stable. The term “storage stable” or “stable” is to be understood as referring to the formulation that is stable under pre-selected storage conditions (see below), including pre-selected storage temperature, pre-selected physical state of the formulation (e.g. fluid/liquid, solid etc.). Stability can be determined by testing the absence of visible aggregations and/or fibrin clots in the formulation under the pre-selected storage condition, for example, when the formulation is in liquid form. Stability can also be determined by measuring the fibrinogen content in the formulation following storage of the formulation, under the pre-selected storage condition, for example, by clotting assay and/or western blot and/or immunoassay. Further, in accordance with the present disclosure, when referring to a stable sealant formulation it is to be understood as one that, upon use, has an effective clotting time irrespective of the formulation's storage conditions, e.g., the sealant formulation clots at essentially the same time period irrespective of whether it was stored at room temperature or at lower temperatures.

In addition, or alternatively, and in accordance with the present disclosure, when referring to a stable sealant formulation it is to be understood as one that, after storage under the pre-selected storage conditions and upon use, the formulation has clotting time of equal to or less than 500 seconds when measured using a Diagnostica Stago START™ clotting machine set to 37° C. e.g. incubating the sample for 60 seconds followed by adding of Unicalibrator and then measuring clotting time at 37° C.

A clotting time of equal to or less than 500 seconds, such as in the range of 100 to 500 seconds, can be beneficial (in the presence of blood or plasma components) e.g. to join tissues (e.g. skin flaps).

In one embodiment the one component sealant is used to prevent reoccurrence of bleeding as it is based on physiological blood clotting components.

In another embodiment, the one component sealant is used to stop or prevent bleeding recurrence within equal to or less than 200 seconds such as within a time range of 100 to 200 seconds.

In some embodiments, the pre-selected storage conditions comprise storage at room temperature (i.e. 20° C.-25° C.) and the sealant formulation is stable for at least 5 minutes, at times for at least 15 minutes, or even for at least 1 hour.

In yet some embodiments, the storage conditions comprise storage of between 2° C.-8° C. and the sealant formulation is stable for at least a day, at least 2, 3, 4, 5, 6 or even 7 days.

In some embodiments, the storage pre-selected conditions comprise storage at equal or below −30° C. and the sealant formulation is stable for at least one month, at times for at least 3, 4, 5, 6, 7, 8, 9, 10, 11, 12, 13, 14, or even 15 months.

The single component sealant formulation comprises at least the following ingredients:

a blood derived fibrinogen concentrate;

a divalent cation; and

Factor XIa.

When referring to “blood derived fibrinogen concentrate” it is to be understood as a composition comprising fibrinogen as well as other blood derived proteins, the concentration of at least fibrinogen being higher than that in whole blood. In some embodiments, the fibrinogen concentrate comprises at least 50% w/w fibrinogen out of the total dry matter.

In some embodiments, the fibrinogen concentrate is other than pure fibrinogen. This is supported by the finding provided herein that when using pure fibrinogen, the formulation spontaneously clots. In other words, based on the findings disclosed herein and without being bound by theory, it has been concluded that components present in the blood derived fibrinogen concentrate are required for the stability of the formulation.

Thus, in accordance with some embodiments, in addition to fibrinogen, the fibrinogen concentrate contains at least one, preferably combination of two or more of fibronectin, albumin, and a residual protein (residual proteins in total up to 1% w/v from the total concentrate composition) selected from immunoglobulin, plasmin(ogen), vWF, Factor VIII, Antithrombin III, and serpine proteins.

Surprisingly, it has been found (as shown in the Examples below) that formulations based on pure fibrinogen were not stable as they spontaneously clotted in the presence of calcium. In contrast, a formulation based on a fibrinogen concentrate, i.e. BAC2 (produced using a cryo-depletion process), was stable in the presence of calcium. Without being bound by theory, this may be due to the presence of additional ingredients aiding in stabilizing the fibrinogen. Thus, it was further concluded that a non-pure fibrinogen (e.g. one that is derived from plasma or blood) is essential for obtaining a suitable single component sealant formulation.

In some embodiments, the fibrinogen concentrate contains at least/not less than 40 g/L (4%) of clottable proteins i.e. proteins comprising mostly fibrinogen and including additional proteins such as Factor XIII—2-9 IU/mL; Fibronectin—0.5-6 mg/mL); and Albumin—9-30 mg/mL.

In some embodiments, the blood derived fibrinogen concentrate comprises cryoprecipitated fibrinogen. In some embodiments, the blood derived fibrinogen concentrate is cryoprecipitated fibrinogen. In the context of the present disclosure, the term “cryoprecipitated fibrinogen” refers to fibrinogen obtained from frozen plasma, the latter prepared from whole blood. A cryoprecipitate can be obtained when frozen plasma is thawed in the cold, typically at a temperature of 0-4° C., resulting in the formation of a precipitate that contains predominantly said fibrinogen. The precipitate can be collected, for example by centrifugation and dissolved in a suitable buffer such as a buffer containing 120 mM sodium chloride, 10 mM trisodium citrate, 120 mM glycine, 95 mM arginine hydrochloride. In some embodiments, the fibrinogen solution comprise additional factors such as, anyone or combination of, factor XIII, factor VIII, fibronectin, von Willebrand factor (vWF), vitronectin, etc.

In some embodiments, the cryoprecipitated fibrinogen is regarded as the biologically active component (BAC) of blood plasma.

There are several types of BAC, all preferably being viral inactivated.

In some embodiments, BAC is a biologically active component that contains tranexamic acid, as an antifibrinolytic agent. BAC containing tranexamic acid is sometimes known by the product name Quixil (Omrix, Israel).

In some other embodiments, BAC is a biologically active component that does not contain tranexamic acid. This is considered a second generation BAC and is referred to in the art as BAC2. During BAC2 preparation, plasminogen (the enzyme precursor of plasmin, which breaks down fibrinogen and fibrin) is removed.

BAC can be prepared as described in U.S. Pat. No. 6,121,232 and/or WO98/033533 the contents of which is incorporated by reference. The composition of BAC can comprise anti-fibrinolytic agents (e.g. tranexamic acid) and arginine hydrochloride. The amount of anti-fibrinolytic agents such as tranexamic acid in the BAC can be from about 80 to about 110 mg/ml.

BAC2 can be prepared as component A according to the disclosure of EP 534 178, the content of which is incorporated herein by reference. For example, component A is prepared from concentrated cryoprecipitate, and undergoes viral inaction by solvent detergent treatment and pasteurization.

In some embodiments, the blood derived fibrinogen concentrate is BAC2, namely, a concentrated viral inactivated cryoprecipitate comprising mainly fibrinogen and is plasminogen-depleted (the removal of plasminogen can be carried out as described in EP 1 390 485). The BAC2 is without tranexamic acid.

In some embodiments, the blood derived fibrinogen concentrate is a by-product of the manufacture process of factor VIII and is selected from the group consisting of acid-precipitate, chill-precipitate, aluminum hydroxide precipitate (see, for example, U.S. Pat. No. 4,455,300, the content of which is incorporated herein by reference), glycine precipitate(see, for example, U.S. Pat. No. 4,297,344, the content of which is incorporated herein by reference), ethanol precipitate, and heparin precipitated paste. Blood derived fibrinogen concentrate as a by-product of the manufacture process of factor VIII is also described in U.S. Pat. No. 9,328,338, the content of which is incorporated herein by reference.

In some embodiments, cryoprecipitated fibrinogen denotes, without being limited thereto, fresh frozen plasma precipitate following centrifugation containing Total protein—30-60 mg/mL; TVC≦1000 CFU/mL; Factor XIII—2-9 IU/mL; Fibronectin—0.5-6 mg/mL; and Colttable Fibrinogen—18-39 mg/mL.

In yet some other embodiments, the blood derived fibrinogen concentrate comprises or is suspended or precipitated Cohn Fraction I, at times, also referred to as Paste I. Cohn fractionation is a process exploiting differences in isoelectric properties of the various plasma proteins and comprises a series of purification steps that involve modifying the pH, ethanol concentration and temperature to separate proteins through precipitation into five “fractions” (I-V). The Cohn process is known also as the cold ethanol precipitation and is described, e.g. in U.S. Pat. No. 2,390,074, and by Cohn et al. (J. Am. Chem. Soc. 68:459, 1945, J. Am. Chem. Soc. 72:465-474, 1950).

Notably, in the context of the present disclosure, when referring to “suspended or precipitated Cohn Fraction I” it is to be understood as encompassing any product of ethanol fractionation whereby at least fibrinogen is precipitated, and not only the Cohn process referred to hereinabove.

Generally, to obtain suspended or precipitated Cohn Fraction I, blood plasma is subjected to ethanol concentration. Specifically, Cohn I precipitate (Fraction I) is obtained from thawed pooled plasma by precipitation at −3° C. to −5° C. and neutral pH at 8-10% ethanol concentration. The paste recovered by centrifugation is considered at times to contains about the same proteins as cry oprecipitate, but to include a fibrinogen yield over 90% [Production of Plasma Proteins for Therapeutic Use; Bertolini, Goss and Curling 2013, page 141].

Any other blood derived fibrinogen concentrate can be used according to the present disclosure as long as it is not pure fibrinogen.

Fibrinogen concentrate can also be obtained commercially. Examples of fibrinogen include, but are not limited to fibrinogen component of EVICEL (i.e. BAC2), fibrinogen component of Tisseel (containing aprotinin, an antifibrinolytic agent).

The plasma derived fibrinogen concentrate can be defined by the amount of clottable proteins therein.

In the context of the present disclosure when referring to a “clottable protein” it is to be understood as encompassing any of the plasma proteins participating in the clotting cascade. As acceptable in the art, clottable proteins include mostly fibrinogen but also some amounts of additional proteins such as Factor XIII, Fibronectin, and Albumin.

The % of “clottable proteins” can be calculated, for example, from clottable and total protein determinations. The “total protein” can be determined by diluting a sample in a solubilizing buffer containing 0.2 mol/l sodium hydroxide and 7 mol/l urea, and comparing the absorbance of the samples at 280 nm with that of a similarly treated house standard calibrated against the World Health Organization International Standard (Fibrinogen Human Concentrate 98/614). The “clottable protein” can be determined by clotting a diluted sample with thrombin (4 IU/ml), washing the clot with phosphate-buffered saline (1:2 PBS:saline) and drying it on filter paper. The dry clots are then dissolved in the solubilizing buffer and the % clottable protein can be determined by comparing the absorbance at 280 nm with that of a similarly treated house standard calibrated against the above-mentioned World Health Organization International Standard.

In some embodiments, the plasma derived fibrinogen concentrate comprises at most 79.8% clottable proteins out of the total amount of protein in the plasma derived fibrinogen concentrate (79.8mg/ml out of 100 mg/ml total proteins).

In one embodiment, the plasma derived fibrinogen concentrate comprises between 65% to 78%, at times, between 68% to 78%, further at times, between 68% to 72% and yet at times about 70% clottable proteins.

In another embodiment, the concentrated fibrinogen comprises total protein in the range of about 96 to about125 mg/ml and clottable protein in the range of about 72 to about 110 mg/ml.

In another embodiment, the concentrated fibrinogen comprises total protein in the range of about 80 to about 120 mg/ml and clottable protein in the range of about 50 to about 90 mg/ml. In some additional or alternative embodiments, the plasma derived fibrinogen concentrate is characterized by a fibronectin relative concentration, i.e. the proportion of fibronectin relative to fibrinogen, defined as the fibronectin to fibrinogen molar ratio.

In some embodiments, the fibronectin to fibrinogen ratio is equal to or above 0.016, or equal to or above 0.065.

In some embodiments, the fibronectin to fibrinogen ratio is equal to or above about 0.068 (i.e. the ratio is greater than 0.068) at times equal to above about 0.078, (i.e. the ratio is greater than 0.078), at times, the ratio is equal to or above 0.1, equal to or above 0.5, equal to or above 1.0, equal to or above 1.5.

In some embodiments, the fibronectin to fibrinogen molar ratio is equal or below 2 (i.e. the ratio is lower than 2), at times below 1.5, at times below 1.0, at times below 0.5, or at times below 0.1.

In some embodiments, the fibronectin to fibrinogen molar ratio is between 0.1 to 2.0.

In yet some other embodiments, the fibronectin to fibrinogen molar ratio is between 0.1 to 0.2.

The plasma derived fibrinogen concentrate can also be characterized by the fibrinogen absolute concentration. In some embodiments, the fibrinogen concentration is between 13 to 85 mg/ml such as 13 to 29 mg/ml, at times 13-21 mg/ml (equivalent to 40-63 μM), at times between 13-17 mg/ml, or 14-18 mg/ml, or 15-19 mg/ml, or 16-20 mg/ml, or 17-21 mg/ml.

The divalent cation, in the context of the present invention includes, without being limited thereto Ca⁺², Mg²⁺, Fe²⁺, Mn²⁺.

In some embodiments, the divalent cation is calcium cation. In some embodiments, the calcium cation is derived from calcium chloride, i.e. the formulation is prepared with calcium chloride.

In some embodiments, the concentration of the CaCl₂ is between about 0.15 to 0.5 mg/ml (equivalent to 3.70-12.50 mM), at times, between 0.15 to 0.25mg/ml (equivalent to 3.70-6.25 mM), at times between 0.25 to 0.5 mg/ml (equivalent to 1.85-12.50 mM).

In some embodiments, preferably when the divalent cation is Ca⁺², the concentration is in the range of about 3.75-7.50 mM.

Turning to Factor XIa, it is to be understood as encompassing the natural, blood derived enzyme, recombinant factor XIa or any analog thereof that is capable of activating the substrate factor IX during hemostasis.

In some embodiments, XIa is in an amount of between 0.01 to 110 μg/ml, 0.11 to 110 μg/ml, such an amount of higher than 0.11 and up to 110 μg/ml.

The sealant formulation can comprise additional ingredients other than the above three recited ingredients.

In some embodiments, the sealant formulation comprises one or more additional ingredients selected from factor XIII, anti-fibrinolytic agents (such as aminocaproic acid (ε-aminocaproic acid), aprotinin and tranexamic acid) antibiotics, stabilizers such as arginine, lysine, fibronectin, von Willebrand factor; RGD peptides; growth factors, cartilage inducing factors, osteoid inducing factors, bone growth factors, collagen growth factors; cytokines; interferons; hormones; therapeutic agents such as antimicrobial agents, anti-inflammatories; anti-cancer drugs; chemotherapy agents; analgesics; interleukins; minerals; molecules which stimulate cell migration, adhesion and/or proliferation; enzymes; neurotrophic factors such as nerve growth factor (NGF); ciliary neurotrophic factor (CNTF), their pharmaceutically acceptable salts, or mixtures thereof, etc. The sealant additives are selected as known to those versed in the art of sealant formulations. A list of possible sealant additional ingredients can be found, inter alia, in U.S. Pat. No. 8,858,969 to Z-Medica “Hemostatic Compositions, Devices and Methods”; and US patent application publication No. 2014/0271610 to Orthovita “Gelatin and Alginate-Based Formulations for Hemostasis”; the contents of which are incorporated herein by reference.

The sealant additives can be isolated from plasma of human beings or mammals or can be recombinant.

In some embodiments, the sealant formulation is depleted/free of vitamin K-dependent clotting zymogens. In some embodiments, the sealant formulation is depleted of Factor IX zymogen. By using the term “depleted” it is to be understood as lacking the said zymogens or including an amount that, if activated, it is ineffective of causing clotting of the sealant formulation.

In some embodiments, the vitamin K-dependent clotting zymogen is selected from FII, FVII, FIX and FX zymogens. As described by Grober U, vitamin K is essential for the g-carboxylation of specific glutamic acid (Glu) residues in a number of vitamin K-dependent proteins. The resultant g-carboxyglutamic acid (Gla) compounds can effect complex binding of calcium ions, leading to a protein conformational change, which is a precondition for its physiological function. In this way, e.g., by means of posttranslational modification, the clotting factors II (prothrombin), VII, IX and X develop from precursors. [Reference Grober U, Reichrath J, Holick MF, Kisters K. Vitamin K: an old vitamin in a new perspective. Dermatoendocrinol. 2015 Jan 21;6(1)].

In some embodiments, the vitamin K-dependent clotting zymogen is prothrombin (FII).

In some embodiments, the sealant formulation is free of thrombin or a functional analog of thrombin. In the context of the present disclosure, being “free of thrombin” (or functional analog thereof) it is to be understood that the formulation may contain residual thrombin, but yet in an amount being ineffective/insufficient of causing coagulation of the sealant formulation. In some embodiments, being free of thrombin (or functional analog thereof) encompasses an amount of not more than 1IU/m1 thrombin. In yet some other embodiments, free of thrombin (or functional analog thereof) refers to lack of externally added thrombin (or functional analog thereof), namely, lack of thrombin (or functional analog thereof) that does not originate from the fibrinogen concentrate.

In the context of the present disclosure, when referring to “thrombin functional analog” it is to be understood as meaning an entity that is capable of at least cleaving fibrinogen to form fibrin.

The amount of thrombin can be determined by ELISA using a Thrombin antibody. Further, or alternatively, the thrombin can be determined by Thrombin activity test using a clotting machine.

The sealant formulation may have various applications, such as for treating bleeding wounds, for sealing physiological leaks, e.g. of cerebrospinal fluids, lymph, bile, gastrointestinal (GI) content air leak from the lungs, for adhering tissues or materials to tissue or as a delivery device substance for drugs or cells, e.g. where the drug or cells are released upon natural degradation of the clot, as discussed above.

For applying the sealant formulation, it may be accommodated within a medical device/applicator. Thus, in accordance with a further aspect of the present disclosure, an applicator comprising (i) a container holding the sealant formulation disclosed herein, and (ii) a sealable opening for delivery therethrough of the sealant formulation.

In some embodiments, the applicator is in a form of a syringe.

In some embodiments, the applicator is in a form of a nebulizer or dispenser that applies the liquid formulation as fine droplets (e.g. by spraying or dripping the formulation onto the target tissue in need of sealing). In some embodiments, e.g. if a large surface area needs to be sealed from bleeding, spraying would be commonly used; if bleeding is occurring from a confined area, e.g. after a biopsy, then dripping would be commonly used. Alternatively, spraying and dripping can be used in the same procedure.

In some embodiments, the opening of the applicator is re-sealable.

The sealant formulation within the barrel may be in liquid or solid (frozen) form. In some embodiments, the applicator is in a form of a syringe holding the sealant formulation in liquid form.

The applicator can be for single use, i.e. to be disposed after all or a portion of the sealant formulation is expelled from the container; or it may be designed for multiple uses such that the applicator's opening is resealed between uses.

In some other embodiments, the applicator is in a form of a wound dressing comprising a support matrix holding the sealant formulation.

The support matrix can be in a form of a bandage, foam pad etc. applied onto the wound. In some embodiments, the support matrix is or comprises a non-woven fabric, such as those used in wet wipes onto which the formulation is absorbed, impregnated, swelled or the like.

The support matrix with the sealant formulation can be contained within a container, preferably sterically sealed, such as in sachets, each being opened prior to use.

The delivery mode of the sealant formulation can be determined based on various considerations, such as, the type/extent of sealing needed, the type/dimensions of opening (requiring sealing) and other considerations as appreciated by the physician.

The sealant formulation is applicable in various medical methods that require biological glues.

In accordance with one aspect, there is thus provided a method for promoting in situ fibrin clot/matrix formation comprising applying to a target tissue the sealant formulation disclosed herein.

In some embodiments, the method is for treating a wound in a subject in need thereof, the method comprises applying onto at least a portion of said wound an amount of the sealant formulation disclosed herein, the amount of said sealant formulation being effective to promote fibrin clot formation in said wound after contact with said wound.

Without being bound by theory, it is the in situ contacting of the sealant formulation with blood (e.g. present at the wounded area) that initiates coagulation cascade, making use of the formulation's ingredients.

Thus, in some embodiments, the method comprises treating a wound, preferably blood containing wound. In some embodiments, the wound is a bleeding wound.

It has been found that once blended with a plasma substitute, clotting occurs within seconds, ˜200 sec (varied according to concentration listed in the tests).

Finally, provided herein, in accordance with another aspect, is a kit comprising a sealant formulation as disclosed herein and, optionally, an applicator. In some embodiments, the kit also comprises instructions for use of the sealant formulation to promote clotting in a wound. The instructions may include steps for preparing the sealant formulation before application onto a wound, and/or steps for applying the sealant formulation onto at least a portion of the wound. In some embodiments, the kit comprises a container including a storage-stable aqueous sealant formulation comprising a blood derived fibrinogen concentrate, a divalent cation and factor XIa, the sealant formulation is stable at a temperature of between 20° C.-25° C. for at least 5 minutes. In some embodiments, the container is in a form of an applicator. In some embodiments, the applicator is configured to facilitate said applying of the sealant formulation on at least a portion of a wound. In some embodiments, the applicator is a syringe. In some embodiments, the kit further comprises an applicator.

In some embodiments, the sealant formulation is in dry or solid form and the instructions comprise wetting the formulation. In this respect, the kit may comprise, at times, a wetting agent, such as saline.

In some further embodiments, the sealant formulation is in dry form and the instructions comprise applying the formulation onto the wound in dry form, to thereby be wetted by excretions from the wound or blood per se, or the step of wetting the dry formulation prior to application onto the wound.

In some embodiments, the kit is provided with a ready-to-use sealant formulation, within the applicator; in some other embodiments, the sealant formulation is supplied within a cartridge or other holding unit/carrier and is introduced into the applicator prior to use.

As used herein, the forms “a”, “an” and “the” include singular as well as plural references unless the context clearly dictates otherwise. For example, the term “a divalent cation” includes one or more such cations which are capable of participating in the clotting cascade.

Further, as used herein, the term “comprising” is intended to mean that the formulation includes the recited ingredients, i.e. the fibrinogen, divalent cation and Factor XIa, but not excluding other additional ingredients. The term “consisting essentially of” is used to define formulations which include the recited ingredients but exclude other elements that may have significance on the clotting cascade. “Consisting of” shall thus mean excluding more than trace elements of other elements. Embodiments defined by each of these transition terms are within the scope of this invention.

Further, all numerical values, e.g. when referring the amounts or ranges of the ingredients constituting the formulation , are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### NON-LIMITING EXAMPLES

In the following non-limiting examples, the following materials have been used. Unless otherwise noted, all materials were stored at 2-8° C.
CaCl₂ (0.025M) obtained from Saifan/Stago (Lot# 10789) and stored at 2-8° C.
Human Albumin 25% aliquots, (RD221014-02) obtained from Talecris (Lot# 110081).
Human Plasma: STA® Unicalibrator (substitute for Standard Plasma, Stago, Cat. Number 00625) which is intended for use as a calibration plasma for the functional assays of coagulation parameters on analyzers of the STA® line suitable to this reagent, including prothrombin time, fibrinogen, factors II, V, VII, VIII, IX, X, XI and XII, protein C and protein S, antithrombin (AT), plasminogen and antiplasmin). TSB Buffer—prepared from 6.05g Tris-base, 9g NaCl and 0.2g Sodium Azide in 1L PuW, pH 7.4 (For “NAPTT”, non-activated partial thromboplastin time being the time to form a clot after the addition of all relevant coagulation factors to the solution. This assay evaluates the presence of activated coagulation factors in plasma and plasma fractions. Whether it's residuals or intended coagulation factors)
Factor XIa (1/5000) aliquots (RD171113-01) obtained from Tarom/HTI (Lot# CC0709) and stored at <−65° C.
BAC2 is a concentrated viral-inactivated cryoprecipitate of human plasma (the cryoprecipitate is typically prepared as described in EP 534,178) which consists mainly of fibrinogen (approx. 85%) and is plasminogen-depleted (the removal of plasminogen is typically carried out as described in EP 1,390,485) and without anti-fibrinolytic agents added.

### Example 1

### Determination of a Minimal Stability Period

The aim of this study was two-fold:
“Storage stability”—To determine the storage stability of a solution containing FXIa and Fibrinogen by incubation of these ingredients for 72 hours and examining whether a clot is formed or not.
“Activity”—to determined, following the 72 hour storage period, the ability of the solution to form a clot as measured by the addition of Unicalibrator and phospholipids (to simulate a physiological environment).

FXIa+BAC2 (Fibrinogen component of the Omrix-Ethicon fibrin sealant), at room temperature or 4° C. were evaluated for 72 hr.

### Stability

Table 1 summarizes the formulations tested. The ratios between BAC2 and Factor XIa varied between 1:1 to 10:1.

TABLE 1
Preparation of formulations:

| | | | | | Final |
|---|---|---|---|---|---|
| | | | | Final | concentration |
| FXIa | BAC2 | Vial No. | Vial No. | concentration | Fibrinogen |
| (μl) | (μl) | (4° C.) | (RT) | FXIa | (BAC2) |
| 100 | 100 | 1 | 5 | 0.082 μg/ml | 8 mg/ml |
| 100 | 200 | 2 | 6 | 0.054 μg/ml | 10 mg/ml |
| 100 | 500 | 3 | 7 | 0.027 μg/ml | 13.3 mg/ml |
| 100 | 1000 | 4 | 8 | 0.001 μg/ml | 14.5 mg/ml |
| — | 100 | 9 (control) | 10 | / | 16 mg/ml |
| | | | (control) | | |

The formulations were introduced into vials, and placed at 4° C. or RT according to Table 1 above for a period of 72 hours. After this short term storage, stability and activity were determined.

No clots were formed in any of the vials irrespective of the FXIa and BAC2 ratios and the storage temperatures.

### Activity

For activity testing, the same formulations of Table 1 were used and these were mixed with a clotting reaction buffer that simulates the physiologic conditions in a bleeding site.

Specifically, the clotting reaction buffer was prepared by adding 400 μl albumin to 2.1 ml of TSB Buffer (described in the Materials).

To each vial of Table 1, 30 μl of the TSB Buffer (with the albumin and Phospholipids) together with all other ingredients, followed by the addition of 30 μof the Unicalibrator.

The vials were then incubated over night at 37° C. No clots formed after overnight incubation at 37° C.

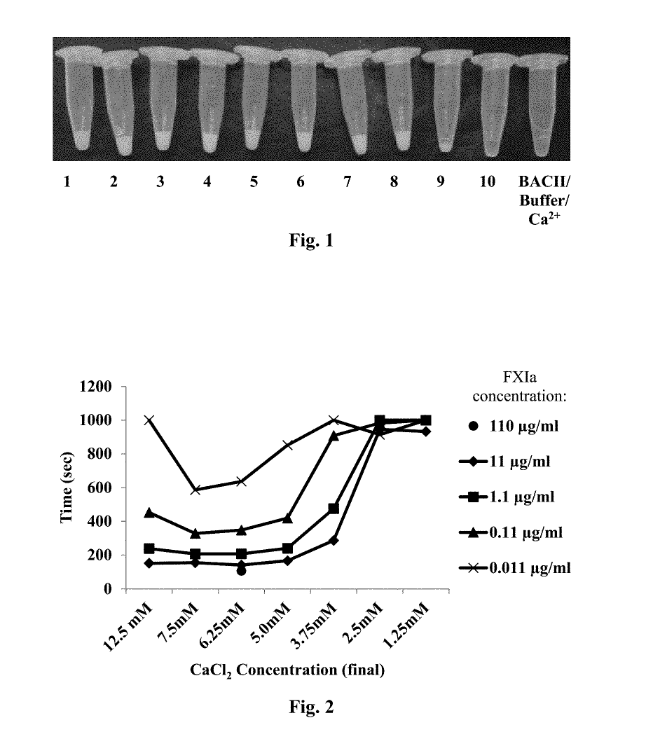

To each vial, 30 μl of Ca²⁺0.025M was then added and the vials were left at RT for 2.5 hr. FIG. 1 shows each vial and the formation of precipitate in the FXIa + BAC2 vial mixtures (vials 1-8) but not in the control vial (vial 10) and vial containing BAC2+Buffer+Ca²⁺ without prior incubation (most right vial). It is noted that the white sediments at the bottom of vial 9 was due to sedimentation of the Ca²⁺.

It was thus concluded that the storing of all ingredients, as a single component formulation, is storage stable and the storage does not affect activity of the ingredients such that once brought into contact with a buffer simulating physiologic conditions and Ca²⁺, a fibrin clot is effectively formed.

### Example 2

### Determination of Essential Reaction Ingredients

The aim of this study was to screen the different formulations in order to determine whether Ca²⁺ and cephalin (substituting platelet phospholipids) are essential for an in vitro reaction.

For the determination of the essential reaction ingredients, combinations of BAC2 (stock concentration of ˜64mg/ml) and FXIa (stock concentration of 0.65 μg/ml “FXIa-0.65” or 0.44 μg/ml “FXIa-0.44”), with or without Ca²⁺ (0.025M) and cephalin (provides the physiological substitute for platelet phospholipids which take part in the coagulation cascade, stroke solution concentration of about 27.4 μg/ml) were tested, as also detailed in Table 2 below. To initiate reaction, the clotting reaction buffer comprising the TSB buffer and albumin (as described in Example 1) was added (“TSB-albumin”)

TABLE 2
Preparation of tubes for formulation testing:

| Test | | FXIa- | FXIa- | | | | TSB- |
|---|---|---|---|---|---|---|---|
| No. | BAC2 | 0.65 | 0.44 | Ca²⁺ | DDW | Cephalin | albumin |
| 1 | 50 μl | 50 μl | / | / | 50 μl | / | 50 μl |
| 2 | 50 μl | 50 μl | / | / | 50 μl | 50 μl | / |
| 3 | 50 μl | 50 μl | / | 50 μl | / | / | 50 μl |
| 4 | 50 μl | 50 μl | / | 50 μl | / | 50 μl | / |
| 5 | 50 μl | / | 50 μl | / | 50 μl | / | 50 μl |
| 6 | 50 μl | / | 50 μl | / | 50 μl | 50 μl | / |
| 7 | 50 μl | / | 50 μl | 50 μl | / | / | 50 μl |
| 8 | 50 μl | / | 50 μl | 50 μl | / | 50 μl | / |

The prepared mixtures were incubated overnight either at 4° C. or at RT. Following incubation, Unicalibrator was added to the vials and the vials were then incubated at 37° C. to test dotting time.

After incubation, the vials were vortexed for 1 second, rolled for 10 min and further incubated as described in Tables 3 (each row represents a single vial) and Table 4 (each row represents a triplicate).

It is already noted that vials containing Cephalin and Ca²⁺ stored over night at RT formed a spontaneous clot.

Following overnight incubation, 50 μl of Unicalibrator was added to some of the samples as indicated in Tables 3 and 4.

All vials were incubated at 37° C. and examined at the following time points: 1, 3, 5, 7, 10, 15, 20, 25, 30, 45, 60 minutes (results summarized in Table 3), and at 0, 1, 2, 3, 4, 5, 7.5, 10, 15, 20, 25, 30, 60 min as shown in Table 3 and 4. Notably, the levels indicated in Table 4 for the different ingredients are calculated respectively to the corresponding International Standards. All ingredients take part in the coagulation cascade.

TABLE 3

| | BAC2 | FXIa | Ca²⁺ | | | | |
|---|---|---|---|---|---|---|---|
| No. | (~16 mg/ml) | (0.163 μg/ml) | (6.25 mM) | Cephalin | Temp. | Unicalibrator | Clot formation |
| 1 | + | + | − | − | 2-8° C. | − | \ |
| | + | + | − | − | 2-8° C. | + | \ |
| | + | + | − | − | RT | − | \ |
| | + | + | − | − | RT | + | \ |
| 2 | + | + | − | + | 2-8° C. | − | \ |
| | + | + | − | + | 2-8° C. | + | \ |
| | + | + | − | + | RT | − | \ |
| | + | + | − | + | RT | + | \ |
| 3 | + | + | + | − | 2-8° C. | − | \ |
| | + | + | + | − | 2-8° C. | + | clot at 5 min |
| | + | + | + | − | RT | − | \ |
| | + | + | + | − | RT | + | clot at 7 min |
| 4 | + | + | + | + | 2-8° C. | − | \ |
| | + | + | + | + | 2-8° C. | + | clot at 3 min |
| | + | + | + | + | RT | − | clot formed overnight |
| | | | | | | | spontaneously |
| | + | + | + | + | RT | + | clot formed overnight |
| | | | | | | | spontaneously |

TABLE 4

| | BAC2 | FXIa | Ca2+ | | | | |
|---|---|---|---|---|---|---|---|
| No. | (~16 mg/ml) | (0.11 μg/ml) | (6.25 mM) | Cephalin | Temp. | Unicalibrator | Clot formation |
| 5 | + | + | − | − | 2-8° C. | − | \ |
| | + | + | − | − | 2-8° C. | + | \ |
| | + | + | − | − | RT | − | \ |
| | + | + | − | − | RT | + | \ |
| 6 | + | + | − | + | 2-8° C. | − | \ |
| | + | + | − | + | 2-8° C. | + | \ |
| | + | + | − | + | RT | − | \ |
| | + | + | − | + | RT | + | \ |
| 7 | + | + | + | − | 2-8° C. | − | \ |
| | + | + | + | − | 2-8° C. | + | clot at 7.5 min |
| | + | + | + | − | RT | − | \ |
| | + | + | + | − | RT | + | clot at 10 min |
| 8 | + | + | + | + | 2-8° C. | − | \ |
| | + | + | + | + | 2-8° C. | + | 2 vials formed at 5 |
| | | | | | | | min. 1 started at 5 |
| | | | | | | | and completed forming |
| | | | | | | | at 7.5 min |
| | + | + | + | + | RT | − | clot formed overnight |
| | | | | | | | spontaneously |
| | + | + | + | + | RT | + | clot formed overnight |
| | | | | | | | spontaneously |

The results presented in Tables 3 and 4 show that without Ca²⁺ there is no polymerization reaction, i.e. no clot is formed.

In the presences of only Ca²⁺, i.e. without human plasma, the formulation remained liquid. The addition to Unicalibrator to the liquid formulation, polymerization took place.

It was thus concluded that Ca²⁺ is essential for clot formation and that cephalin should not be added to the formulation since it causes spontaneous clotting. Specifically, according to these results, the preferable formulation should contain, at minimum BAC2, FXIa, and Ca²⁺.

### Example 3

### Determination of the Effect of Ca2+ and FXIa Concentration on the Formula

The aim of this study was to determine the effect of different concentrations of Ca2+ and FXIa ingredients. To this end, serial dilutions were performed for both ingredients and clotting time following the addition of Unicalibrator was measured. The clotting time of formulation containing BAC2, Ca²⁺ and Unicalibrator (without adding FXIa) was used as a baseline (control).

Reaction mixtures were prepared as follows:

For baseline determination: 150 μl Buffer A (used to dilute the FXIa and comprises 2.4 g HEPES, 4.38 g NaCl and 0.5 g Bovine Serum Albumin, into 400 ml purified water, and pH was adjusted to 7.4) 150 μl BAC2, and 150 μl CaCl₂ were mixed.

For determination of FXIa and Ca²⁺ concentrations: 10 μl Stock FXIa were diluted with 990 μl of Buffer A (dil. 1/10), and rolled (40 RPM) for 10 min. Then, 0.5 ml of this preparation were mixed with 4.5 ml Buffer A (final volume 5 ml), and rolled for 5 min. Serial 1:10 dilutions of 0.5 ml (previous dilution)+4.5ml buffer A were repeated to achieve all dilutions. The various FXIa dilutions are summarized in Table 5.

TABLE 5
FXIa dilution concentrations:
Dilution

| Dilution factor | Concentration | Final concentration |
|---|---|---|
| 1/10 | 440 μg/ml | 110 μg/ml |
| 1/100 | 44 μg/ml | 11 μg/ml |
| 1/1000 | 4.4 μg/ml | 1.1 μg/ml |
| 1/10000 | 0.44 μg/ml | 0.11 μg/ml |
| 1/100000 | 0.044 μg/ml | 0.011 μg/ml |

Test samples were prepared according to Table 6 below. Each vial contained:
1) 210 μl BAC2 (Total protein concentration 80-120 mg/ml; clottable (fibrinogen concentration 55-85 mg/ml);
210 μl FXIa (concentration is added according to Table 6);
3) 210 μl CaCl₂ (concentration is added according to Table 6).

The obtained final concentration of proteins from BAC2 was 26.7 to 40 mg/ml; and final concentration of clottable fibrinogen was 18.33 to 28.33 mg/ml.

Once all ingredients were added, the vials were rolled for 5 min. From each sample, a volume of 150 μl were added into each designated clotting machine cuvette (Diagnostica stago START). Four replicates were used for each sample.

Clotting time was measured using a clotting machine (Diagnostica stago START). Reaction time is measured starting with the addition of 50 μl of Unicalibrator (preheated to 37° C.). Measurement were recorded either as seconds to form a clot (when a clot is formed in less than 960 seconds) or as >960 seconds, if a clot is not formed by this time.

The time to clot formation (in seconds) according to the different FXIa and Ca²⁺ concentrations is presented in Table 7 below. It is noted that time measurement began with the addition of Unicallibrator. The baseline (Control—no FXIa test) was 768.1±15.1 seconds for polymerization.

TABLE 6
Ca²⁺ and FXIa dilution set:

| | | FXIa | | | | |
|---|---|---|---|---|---|---|
| | | Dilution: | | | | |
| | | 1:10 | 1:100 | 1:1000 | 1:10000 | 1:100000 |
| | | Concentration: | | | | |
| Ca²⁺ | | 440 μg/ml | 44 μg/ml | 4.4 μg/ml | 0.44 μg/ml | 0.044 μg/ml |
| Concentration: | | Final concentration: | | | | |
| [M] | | 110 μg/ml | 11 μg/ml | 1.1 μg/ml | 0.11 μg/ml | 0.011 μg/ml |
| 0.05 | 12.5 mM | \ | 6 | 12 | 18 | 24 |
| 0.03 | 7.5 mM | \ | 25 | 26 | 27 | 28 |
| 0.025 | 6.25 mM | 29 | 1 | 7 | 13 | 19 |
| 0.02 | 5.0 mM | \ | 2 | 8 | 14 | 20 |
| 0.015 | 3.75 mM | \ | 3 | 9 | 15 | 21 |
| 0.01 | 2.5 mM | \ | 4 | 10 | 16 | 22 |
| 0.005 | 1.25 mM | \ | 5 | 11 | 17 | 23 |

Numbers in the table represent the order in which samples were prepared.

TABLE 7
Ca²⁺ and FXIa dilutions clotting time (seconds):

| | FXIa | | | | |
|---|---|---|---|---|---|
| | Final concentration: | | | | |
| Ca²⁺ | 110 μg/ml | 11 μg/ml | 1.1 μg/ml | 0.11 μg/ml | 0.011 μg/ml |
| 12.5 mM | \ | 151.5 ± 8.6 | 238.6 ± 9.3 | 452.7 ± 16.3 | >960 |
| 7.5 mM | \ | 154.8 ± 9.6 | 206.7 ± 16.9 | 328.3 ± 67.1 | 586.5 ± 207.9 |
| 6.25 mM | 106.4 ± 0.6 | 140.9 ± 9.7 | 207.2 ± 17.8 | 348.2 ± 74.5 | 636.7 ± 237.0 |
| 5.0 mM | \ | 166.3 ± 8.8 | 240.2 ± 9.3 | 419.4 ± 57.0 | 851.8 ± 303.7 |
| 3.75 mM | \ | 286.7 ± 4.4 | 475.5 ± 3.3 | >960 | >960 |
| 2.5 mM | \ | >960 | >960 | >960 | >960 |
| 1.25 mM | \ | >960 | >960 | >960 | >960 |

The results in Table 7 above are also presented in FIG. 2 and show that CaCl₂ concentrations on different FXIa concentrations curing (clot formation) rate. The horizontal line represents the baseline (no-FXIa test results) and for display purposes, results>960sec were represented as 1000 in this FIG. 2.

Only one sample of the highest FXIa concentration was used (final dilution of 110 μg/ml) and is referred to as the fastest clotting time. In other words, for all dilutions of FXIa (other than the highest concentration) a matrix of CaCl₂ concentrations were tested and for the highest concentration of FXIa, only one concentration of CaCl₂ was applied. This point was used as the reference for the fastest clotting time. Clotting of both FXIa dilutions 1:100 and 1:1000 (11 μg/ml and 1.1 μg/ml accordingly) was similar and change of Ca²⁺ concentration between the range of 12.5-5.0 mM (final) had no effect on clotting time.

A correlation between Ca²⁺ concentration and time to clot formation was observed as a decrease in Ca²⁺ concentration in the reaction resulted in slower clot formation.

Diluting FXIa to 1:10000 (110 ng/ml final concentration) resulted in slower clot formation compared to the higher FXIa concentrations. For this lower FXIa concentration, a stronger effect is observed when Ca²⁺ concentrations are decreased. Additionally, Ca²⁺ concentration of 12.5 mM showed slight lower effect on the curing rate.

Diluted FXIa 1:100000 (11 ng/ml) displayed a much slower curing rate and the effects of the Ca²⁺ concentration show the same trend as in the 110 ng/ml dilution.

Based on the above results it was concluded that the FXIa dilutions with working potential are 1/100 (11 μg/ml), 1/1000 (1.1 μg/ml), 1/10000 (110 ng/ml). The preferable working Ca²⁺ concentration is 6.25 mM (final concentration. using Ca²⁺ stock of 0.025M).

In order to achieve a clinically relevant clot forming rate of less than 960 seconds an estimated time value as deduced from current literature through the use of existing hemostatic products (Topical hemostatic agents. Seyednejad et al 2008), the following formulations should be used (FXIa range 0.01- 110 ug/ml and Ca²⁺ range 3.75-12.5 mM).

### Example 4

### Determination of Short and Long Term Stability of the Formulation

The aim of this study was to determine the stability of the formulation at 3 different temperatures for a period of up to 1 year. In order to determine the short and long term stability of the single component formulations, 3 different FXIa concentrations were incubated at 3 different temperatures (RT, 2-8° C. and −30° C.). Following the incubation period at the selected temperature, each vial was heated to 37° C. followed by addition of Unicalibrator. Time to clotting was then recorded using a clotting machine.

Specifically, 36 samples were prepared as follows:

12 samples incubated at 2-8° C.

12 samples incubated at RT.

12 samples incubated at −30° C.

For each incubation temperature, duplicates (2 vials) were marked for 7 incubation periods of:

1 day (1 d), 1 week (1 w), 2 weeks (2 w), 1 month (1 m), 2 months (2 m), 6 months (6 m), and 15 months (15 m).
Volumes used:

BAC2—120 l*3 (treatments)*36 (samples)=12.96 ml

CaCl₂—120 μl*3 (treatments)*36 (samples)=12.96ml

FXIa—120 μl*1 (dilution)*36 (samples)=4.32ml (for each dilution)

Dilutions for FXIa were prepared from 1:10 dilution stock (Stock concentration of 440 μg/ml):
1/100: 275 μl (1/10)+2.475 ml (buffer A)→4 2.75 ml (1/100)+2.5 ml (1/100, prey. dilution)
1/1000: 400 μl (1/100)+3.6 ml (buffer A)→4.0 ml (1/1000)+850 μl (1/1000, prey. dilution)
1/10,000: 400 μl (1/1000)+3.6 ml (buffer A)→4.0 ml (1/10,000)+850 l (1/10,000, prey.

dilution)

Each vial was supplemented with: 200 μl BAC2, 200 μl CaCl₂ (0.025M stock concentration before mixing), 200 μl FXIa (according to defined test). Vials were then vortexed for 1 sec, rolled for 10 min followed by incubation according to the selected treatment. Two vials of each treatment (independent duplicates) were prepared. The resulted dotting times (in seconds) are presented in Tables 8A-8C.

TABLE 8A
Clotting time for 11 μg/ml (final concentration) FXIa:

| | | |
|---|---|---|
| RT | 2-8° C. | −30° C. |

| | |
|---|---|
| Immediate | 140.9 ± 6.4 |

| | | | |
|---|---|---|---|
| Day 1 | 223.3 ± 2.2 | 125.1 ± 1.1 | 130.7 ± 3.5 |
| 1 week | 294.7 ± 11.7 | 205.6 ± 10.8 | 144.3 ± 4.6 |
| 2 weeks | 341.9 ± 3.1 | 287.9 ± 3.5 | 143.9 ± 2.1 |
| 1 month | 467.1 ± 9.1 | 361.1 ± 9.1 | 119.4 ± 5.7 |
| 2 month | 639.9 ± 23.8 | 531.9 ± 31.2 | 157.1 ± 3.2 |
| 6 month | >960 | 605.7 ± 116.4 | 134.4 ± 3.3 |
| 15 month | NA | NA | 107.2 ± 8.8 |

TABLE 8B
Clotting time for 1.1 μg/ml (final concentration) FXIa:

| | | |
|---|---|---|
| RT | 2-8° C. | −30° C. |

| | |
|---|---|
| Immediate | 207.2 ± 4.6 |

| | | | |
|---|---|---|---|
| Day 1 | 408.7 ± 8.2 | 204.8 ± 0.8 | 193.2 ± 1.4 |
| 1 week | 558.7 ± 2.6 | 371.4 ± 9.8 | 209.8 ± 4.9 |
| 2 weeks | 637.9 ± 29.5 | 499.5 ± 9.4 | 199.6 ± 2.6 |
| 1 month | 812.4 ± 9.6 | 596.1 ± 41.4 | 179.8 ± 5.7 |
| 2 month | >960 | 662.3 ± 19.8 | 232.1 ± 6.0 |
| 6 month | >960 | 809.3 ± 56.6 | 216.4 ± 11.7 |
| 15 month | NA | NA | 189.7 ± 10.5 |

TABLE 8C
Clotting time for 0.11 μg/ml (final concentration) FXIa:

| | | | |
|---|---|---|---|
| Final FXIa | | | |
| Concentration: | | | |
| 0.11 μg/ml | RT | 2-8° C. | −30° C. |

| | |
|---|---|
| Immediate | 348.2 ± 9.3 |

| | | | |
|---|---|---|---|
| Day 1 | 667.8 ± 14.5 | 334.6 ± 11.4 | 307.6 ± 5.1 |
| 1 week | >960 | 693.1 ± 12.7 | 337.9 ± 9.2 |
| 2 weeks | >960 | 809.8 ± 60.7 | 332.8 ± 11.3 |
| 1 month | >960 | 892.2 ± 11.0 | 282.2 ± 0.7 |
| 2 month | >960 | >960 | 336.8 ± 35.7 |
| 6 month | >960 | >960 | 339.1 ± 13.9 |
| 15 month | NA | NA | 298.5 ± 14.2 |

The results presented in Tables 8C show that at a concentration of 0.11 ug/ml FXIa is stable when stored at −30° C. for at least 15 months, and is stable when stored at 2-8° C. for at least 1 day.

The results presented in Tables 8B show that at a concentration of 1.1 ug/ml FXIa is stable when stored at −30° C. for at least 15 months, is stable when stored at 2-8° C. for at least two weeks, and is stable when stored at RT for at least a day.

The results presented in Tables 8A show that at a concentration of 11.1 ug/ml FXIa is stable when stored at −30° C. for at least 15 months, and is stable when stored at 2-8° C. for up to two months, and is stable when stored at RT for up to 1 month.

### Example 5

### Determination of Short and Long Term Stability of the Formulation Using Pure Fibrinogen

The aim of this study was to determine the effect of pure fibrinogen (Human Fibrinogen Plasminogen Depleted (Tarom Applied technologies. 100.00% clottable) on the short and long term stability of the formulations with and without calcium. To this end, two different FXIa concentrations were incubated at two different temperatures (RT, and 2-8° C.). Following the incubation period at the selected temperature, each vial was heated to 37° C. followed by addition of Unicalibrator. Time to clotting (in seconds) was then recorded using a clotting machine.

Specifically, lyophilized fibrinogen was reconstituted using 15ml DDW pre-heated to 37° C. and mixed at 37° C. until completely dissolved.
FXIa 11 μg/ml: 900 l (1/10)+8.1 ml (Buffer A)
FXIa 1.1 μg/ml: 900 μl (1/100)+8.1 ml (Buffer A)

The tests included FXIa concentration of 11 μg/ml, 1.1 μg/ml. In total 33 samples of each test were prepared as follows:
3 samples for Immediate test.

Incubate out of each treatment: 15 samples overnight at 2-8° C.
15 samples overnight at RT.

Each vial was supplemented with: 140 μl fibrinogen, 140 μl FXIa, and 140 μl CaCl₂ (0.025M) to the designated vials (according to test). Vials were then vortexed for 1 sec, rolled for 10 min followed by incubation according to the selected treatment. Three vials of each treatment (independent duplicates) were prepared.

The stability test parameters were as follows:

Fibrinogen concentration: ˜22mg/ml (during stability period) and ˜16.7mg/ml during clotting time test

CaCl₂ concentration: ˜8 mM (during stability period) and 6.25 mM during clotting time test

FXIa dilutions (and concentrations during clotting time test): 1:100 (11 μg/ml), and 1:1000 (1.1 μg/ml).

Temperature RT , 2-8° C. .

Time points: 1 day, 1 week, 2 weeks, 1 month, and 2 months.

The clotting time is summarized in Tables 9A-9C.

TABLE 9A
Clotting time after RT incubation

| Final FXIa | Time | | | |
|---|---|---|---|---|
| Concentration: | points | Immediate | 1 day | 1 week |
| 11 μg/ml | +Ca | 216.6 ± 125.7 | 258.4 ± 13.6 | / |
| | −Ca | 205.5 ± 7.6 | 239.8 ± 3.2 | / |
| 1.1 μg/ml | +Ca | 273.2 ± 47.9 | 365.4 ± 27.4 | / |
| | −Ca | 294.3 ± 16.8 | 416.8 ± 8.8 | / |

TABLE 9B
Clotting time after 2-8° C. incubation

| Final FXIa | Time | | | |
|---|---|---|---|---|
| Concentration: | points | Immediate | 1 day | 1 week |
| 11 μg/ml | +Ca | 216.6 ± 125.7 | 227.1 ± 3.2 | / |
| | −Ca | 205.5 ± 7.6 | 235.8 ± 10.4 | 305.2 ± 14.9 |
| 1.1 μg/ml | +Ca | 273.2 ± 47.9 | 310.4 ± 7.6 | / |
| | −Ca | 294.3 ± 16.8 | 326.3 ± 6.1 | 448.4 ± 5.6 |

The results presented in Table 9A show that at both concentrations of 1.1 and 11 ug/ml FXIa with and without calcium the sealant is stable when stored at RT for less than a week. Within a week, the sampled clotted spontaneously in the presence or absence of calcium.

The results presented in Table 9B show that at both concentrations of 1.1 and 11 μg/ml FXIa the sealant was stable for less than 1 week with calcium. In one week the sealant, only in the presence of calcium, clotted spontaneously.

A comparison between the results in Tables 8 and 9 with or without storage show that the sealant composition comprising the BAC2 component is more active as compared to the composition comprising the pure fibrinogen.

1. A storage-stable aqueous sealant formulation comprising a blood derived fibrinogen concentrate, a divalent cation and factor XIa, the sealant formulation is stable at a temperature of between 20° C.-25° C. for at least 5 minutes.

2. The sealant formulation of claim 1, wherein said divalent cation is a calcium cation optionally, provided by calcium chloride optionally, in an amount of between 0.15 to 0.5 mg/ml.

3. The sealant formulation of claim 1, comprising fibronectin at a relative concentration of fibronectin:fibrinogen of more than 0.078 such as at a relative concentration of fibronectin: fibrinogen of between 0.1 to 0.2.

4. The sealant formulation of claim 1, comprising clottable proteins in an amount that is lower than 79.8% out of the total amount of proteins in said formulation, the total amount excludes said factor XIa.

5. The sealant formulation of claim 1, that is in liquid form.

6. The sealant formulation of claim 1, that is in solid frozen form.

7. The sealant formulation of claim 1, wherein said fibrinogen is in an amount of between 13 to 85 mg/ml such as 13 to 29 mg/ml.

8. The sealant formulation of claim 1, wherein said factor XIa is in an amount of between 0.01 to 110 g/ml, such as an amount of between 0.11 to 110 g/ml, ans such as an amount higher than 0.11 and up to 110 μg/ml.

9. The sealant formulation of claim 1, that is free of thrombin.

10. The sealant formulation of claim 1, that is stable at a temperature of between 2° C.-8° C. for at least two weeks.

11. The sealant formulation of claim 1, that is stable at a temperature of −30° C. for at least 15 months.

12. The sealant formulation of claim 1, that is free of vitamin K dependent clotting zymogens.

13. An applicator comprising (i) a container holding a sealant formulation comprising a storage-stable aqueous sealant formulation comprising a blood derived fibrinogen concentrate, a divalent cation and factor XIa, the sealant formulation is stable at a temperature of between 20° C.-25° C. for at least 5 minutes; and (ii) a re-sealable opening for delivery therethrough of the sealant formulation.

14. The applicator of claim 13, that is in a form of a syringe holding said sealant formulation in liquid form.

15. A wound dressing comprising a support matrix holding a sealant formulation, the sealant formulation comprises storage-stable aqueous sealant formulation comprising a blood derived fibrinogen concentrate, a divalent cation and factor XIa, the sealant formulation is stable at a temperature of between 20° C.-25° C. for at least 5 minutes.

16. A method for promoting the formation of a fibrin clot, the method comprises contacting blood with a sealant formulation comprising storage-stable aqueous sealant formulation comprising a blood derived fibrinogen concentrate, a divalent cation and factor XIa, the sealant formulation is stable at a temperature of between 20° C.-25° C. for at least 5 minutes.

17. A method of treating a wound in a subject in need thereof, the method comprises applying onto at least a portion of said wound an amount of a sealant formulation comprising a storage-stable aqueous sealant formulation comprising a blood derived fibrinogen concentrate, a divalent cation and factor XIa, and is stable at a temperature of between 20° C.-25° C. for at least 5 minutes, the amount of said sealant formulation is effective to promote clotting in said wound when brought into contact with said wound.

18. A kit comprising a container including a storage-stable aqueous sealant formulation comprising a blood derived fibrinogen concentrate, a divalent cation and factor XIa, the sealant formulation is stable at a temperature of between 20° C.-25° C. for at least 5 minutes.
